(19) United States
(12) Patent Application Publication
(10) Pub. No.: US 2025/0235514 A1
(43) Pub. Date: Jul. 24, 2025

(54) POPLYPEPTIDES WITH LYSOPHOSPHATIDYLCHOLINE (LPC) DEGRADING ACTIVITY

(71) Applicant: KERTH CORPORATION, MILPITAS, CA (US)

(72) Inventors: Liang-Yin Ke, MILPITAS, CA (US); Shu-Hsuan Liu, MILPITAS, CA (US); Hua-Chen Chan, MILPITAS, CA (US); Shi Hui Law, MILPITAS, CA (US); Hsiu-Chuan Chan, MILPITAS, CA (US); Guan-Ming Ke, MILPITAS, CA (US)

(21) Appl. No.: 19/005,778

(22) Filed: Dec. 30, 2024

Related U.S. Application Data
(60) Provisional application No. 63/622,829, filed on Jan. 19, 2024.

(51) Int. Cl. A61K 38/46; A61P 9/10; C12N 9/16

(52) U.S. Cl. CPC A61K 38/465; A61P 9/10; C12N 9/16; C12Y 301/04038

(57) ABSTRACT

The present invention related to a new polypeptide DeLCify with lysophosphatidylcholine (LPC)-degrading activity, which is effective in treating a disease or a disorder related to LPC, such as a cardiovascular disease or a neurodegenerative disease. Also provided is a method for the treatment of a disease or disorder related to LPC using the polypeptide DeLCify and a pharmaceutical composition comprising the polypeptide DeLCify.

### CROSS REFERENCE TO RELATED APPLICATION

The present application claims the priority benefit of U.S. Provisional Application Ser. Number U.S. 63/622,829, filed on Jan. 19, 2024, the disclosure of which is hereby incorporated by reference herein in its entirety.

### FIELD OF THE INVENTION

The present invention provides a new polypeptide with lysophosphatidylcholine (LPC) degrading activity, which is effective for treating a cardiovascular disease.

### STATEMENT REGARDING SEQUENCE LISTING

The sequence listing associated with this application is provided in text format in lieu of a paper copy and is hereby incorporated by reference into the specification. The name of the XML file containing the sequence listing is sl.xml. The XML file is 10,819 bytes; was created on Dec. 30, 2024; and is being submitted electronically via EFS-Web.

### BACKGROUND

Atherosclerotic cardiovascular disease (ASCVD) is a class of diseases comprising of atherosclerosis, diabetes, metabolic syndrome, myocardial infarction, and angina. According to the World Health Organization, CVDs account for 17.7 million deaths (31% of all global deaths) each year and are now the leading cause of death worldwide. (Khan M et al. Development, testing, and implementation of a training curriculum for nonphysician health workers to reduce cardiovascular disease. Global heart. 2018 Jun. 1; 13(2):93-100.) Although low-density lipoprotein cholesterol (LDL-C) is an established risk factor for myocardial infarction, a national study has shown that nearly 75% of patients hospitalized for a heart attack had LDL-C levels within normal ranges outlined by national cholesterol guidelines. Specifically, these patients had LDL-C levels that met current guidelines, and close to half of them had LDL-C levels classified in guidelines as optimal (less than 100 mg/dL). In this study, 21% of the patients were taking lipid-lowering medications before admission. (Sachdeva A et al. Lipid levels in patients hospitalized with coronary artery disease: an analysis of 136,905 hospitalizations in Get With The Guidelines. American heart journal. 2009 Jan. 1; 157(1):111-7.) Furthermore, many middle-aged individuals without any cardiovascular risk factors have atherosclerosis. LDL-C, even at levels currently considered normal, is independently associated with the development of early systemic atherosclerosis in the absence of major cardiovascular risk factors. (Fernández-Friera L et al. Normal LDL-cholesterol levels are associated with subclinical atherosclerosis in the absence of risk factors. Journal of the American College of Cardiology. 2017 Dec. 19; 70(24):2979-91.)

It was reported previously that oxidized LDLs played a proatherogenic role in the cell lines and animal models of atherosclerosis. In particular, oxidized LDL causes atherosclerosis through: (a) chemotactic and proliferating actions on monocytes/macrophages, inciting their transformation into foam cells; (b) stimulation of smooth muscle cells (SMCs) recruitment and proliferation in the tunica intima; (c) eliciting endothelial cells, SMCs, and macrophages apoptosis with ensuing necrotic core development. The evident association between oxidized LDLs and cardiovascular events points towards a role of oxidized LDLs in atherosclerotic plaque development and destabilization. (Maiolino G et al. The role of oxidized low-density lipoproteins in atherosclerosis: the myths and the facts. Mediators of inflammation. 2013 Oct. 3; 2013.)

Lysophosphatidylcholine (LPC) is the major bioactive lipid component of oxidized LDL, thought to be responsible for many of the inflammatory effects of oxidized LDL described in both inflammatory and endothelial cells. LPC is formed by cleavage of phosphatidylcholine by phospholipase A2 (PLA2). LPC plays a biological role by binding to G protein-coupled receptors and toll-like receptors. LPC can induce the migration of lymphocytes and macrophages, increase the production of pro-inflammatory cytokines, induce oxidative stress, and promote apoptosis, which can aggregate inflammation and promote the development of diseases. The effects of LPC on endothelial cells, vascular smooth muscle cells and arteries play a vital role in the progression of atherosclerosis and other cardiovascular diseases. In addition, LPC has various roles in inflammatory and infectious diseases. In diabetes, LPC can induce insulin resistance. The concentration of LPC affects the invasion, metastasis and prognosis of different tumours. Therefore, targeting LPC and lipid metabolism might be a potential therapeutic target for inflammation-related diseases. (Liu P et al. The mechanisms of lysophosphatidylcholine in the development of diseases. Life sciences. 2020 Apr. 15; 247:117443; Law, Shi-Hui, et al. “An updated review of lysophosphatidylcholine metabolism in human diseases.” International journal of molecular sciences 20.5 (2019): 1149.)

LPC is an important component of oxLDL and emphasizes the potential role of phospholipase A2 in atherosclerosis. It was reported that LPC was involved in the antigenicity of oxidized LDL (Ruihua Wu et al. Lysophosphatidylcholine Is Involved in the Antigenicity of Oxidized LDL: Arteriosclerosis, Thrombosis, and Vascular Biology. 1998; 18:626-630; Pantazi, Despoina, Constantinos Tellis, and Alexandros D. Tselepis. “Oxidized phospholipids and lipoprotein-associated phospholipase A2 (Lp-PLA2) in atherosclerotic cardiovascular disease: An update.” Biofactors 48.6 (2022): 1257-1270.)

While PLA2 inhibition to decrease production of LPC seems to be a plausible therapeutic target in preventing cardiovascular morbidity and mortality, studies have shown otherwise. In a 2014 randomized clinical trial, inhibition of secretory PLA2 and lipoprotein-associated phospholipase A2 with varespladib and darapladib, respectively, did not reduce cardiovascular ischemic complications and resulted in an excess rate of myocardial infarction and the composite of cardiovascular mortality, myocardial infarction, and stroke in patients with acute coronary syndrome. These results can be attributed to the increase in platelet-activating factor caused by the inhibition of secretory PLA2 and lipoprotein-associated phospholipase A2. (Nicholls S J et al. Varespladib and cardiovascular events in patients with an acute coronary syndrome: the VISTA-16 randomized clinical trial. Jama. 2014 Jan. 15; 311(3):252-62; Talmud, Philippa J., and Michael V. Holmes. “Deciphering the causal role of sPLA2s and Lp-PLA2 in coronary heart disease.” Arteriosclerosis, thrombosis, and vascular biology 35.11 (2015): 2281-2289.)

It is still desirable to develop a new approach for treating a cardiovascular disease, particularly an atherosclerotic cardiovascular disease.

### SUMMARY

Accordingly, the present invention provides a new polypeptide deLCify having activities for degradation of lysophosphatidylcholine (LPC) which is effective in treating a disease or disorder related to LPC, such as a cardiovascular disease, particularly an atherosclerotic cardiovascular disease, or a neurodegenerative disease.

In one aspect, the present invention provides a new polypeptide deLCify having activities for degradation of LPC.

In the invention, LPC includes the carbon chains containing more than 16 carbons, including for example, LPC(16:0), PC(18:0), LPC(20:0), LPC(22:0) or LPC(24:0).

In one embodiment of the invention, the polypeptide deLCify is a polypeptide comprising the amino acid sequence set forth in SEQ ID NO: 1, or a functional fragment, a variant, a modified polypeptide thereof.

According to the invention, SEQ ID NO: 1 consists of the amino acid sequence below:

(SEQ ID NO: 1)
SGYHIGVGRADCTGQVADINLMGYGKSGQNAQGILTRLYSRAFIMAEPD
GSNRTVFVSIDIGMVSQRLRLEVLNRLQSKYGSLYRRDNVILSGTHTHS
GPAGYFQYTVFVIASEGFSNQTFQHMVTGILKSIDIAHTNMKPGKIFIN
KGNVDGVQINRSPYSYLQNPQSERARYSSNTDKEMIVLKMVDLNGDDLG
LISWFAIHPVSMNNSNHLVNSDNVGYASYLLEQEKNKGYLPGQGPFVAA
FASSNLGDVSPNILGPRCINTGESCDNANSTCPIGGPSMCIAKGPGQDM
FDSTQIIGRAMYQRAKSKTCKPALGYSFAAGTIDGVGGLNFTQGKTEGD
PFWDTIRDQILGKPSEEIKECHKPKPILLHTGELSKPHPWHPDIVDVQI
ITLGSLAITAIPGEFTTMSGRRLREAVQAEFASHGMQNMTVVISGLCNV
YTHYITTYEEYQAQRYEAASTIYGPHTLSAYIQLFRNLAKAIATDTVAN
L.

In one example of the invention, the polypeptide deLCify may be a polypeptide comprising the amino acid sequence set forth in SEQ ID NO: 1, and an additional fragment at N-terminus:

(SEQ ID NO: 2)
MAKRTFSNLETFLIFLLVMMSAITVALLSLLFITSGTIENHKDLGGHFF
STTQSPPATQGSTAAQRSTATQHSTATQSSTATQTSPVPLTPESPLFQN
F.

In another example of the invention, the polypeptide deLCify may be a polypeptide comprising the amino acid sequence set forth in SEQ ID NO: 1, and an additional fragment at N-terminus:

(SEQ ID NO: 3)
MPLFQMPLFQNF.

In one example of the invention, the polypeptide deLCify may be a polypeptide comprising the amino acid sequence set forth in SEQ ID NO: 1, and an additional fragment at C-terminus:

(SEQ ID NO: 4)
SRGPEPPFFKQLIVPLIPSIVDRAPKGRTFGDVLQPAKPEYRVGEVAEV
IFVGANPKNSVQNQTHQTFLTVEKYEATSTSWQIVCNDASWETRFYWHK
GLLGLSNATVEWHIPDTAQPGIYRIRYFGHNRKQDILKPAVILSFEGTS
PAFEVVTI.

In another example of the invention, the polypeptide deLCify may be a polypeptide comprising the amino acid sequence set forth in SEQ ID NO: 1, and an additional fragment at C-terminus:

(SEQ ID NO: 5)
SRGPEPPFFKQLIVPLIPSIVDRAPKGRTFGDVLQPAKPEYRVGEVAEV
IFVGANPKNSVQNQTHQTFLTVEKYEATSTSWQIVCNDASWETRFYWHK
GLLGLSNATVEWHIPDTAQPGIYRIRYFGHNRKQDILKPAVILSFEG.

In specific examples of the invention, the polypeptide DeLCify is

(i)
DeLCify-1 (Length 745, Mass (Da) 81,718) having
the amino acid sequence below:
(SEQ ID NO: 6)
MAKRTFSNLETFLIFLLVMMSAITVALLSLLFITSGTIENHKDLGGHFF
STTQSPPATQGSTAAQRSTATQHSTATQSSTATQTSPVPLTPESPLFQN
FSGYHIGVGRADCTGQVADINLMGYGKSGQNAQGILTRLYSRAFIMAE
PDGSNRTVFVSIDIGMVSQRLRLEVLNRLQSKYGSLYRRDNVILSGTH
THSGPAGYFQYTVFVIASEGFSNQTFQHMVTGILKSIDIAHTNMKPGK
IFINKGNVDGVQINRSPYSYLQNPQSERARYSSNTDKEMIVLKMVDL
NGDDLGLISWFAIHPVSMNNSNHLVNSDNVGYASYLLEQEKNKGYL
PGQGPFVAAFASSNLGDVSPNILGPRCINTGESCDNANSTCPIGGPSM
CIAKGPGQDMFDSTQIIGRAMYQRAKSKTCKPALGYSFAAGTIDGVG
GLNFTQGKTEGDPFWDTIRDQILGKPSEEIKECHKPKPILLHTGELSKP
HPWHPDIVDVQIITLGSLAITAIPGEFTTMSGRRLREAVQAEFASHGM
QNMTVVISGLCNVYTHYITTYEEYQAQRYEAASTIYGPHTLSAYIQLF
RNLAKAIATDTVANLSRGPEPPFFKQLIVPLIPSIVDRAPKGRTFGDVL
QPAKPEYRVGEVAEVIFVGANPKNSVQNQTHQTFLTVEKYEATSTSW
QIVCNDASWETRFYWHKGLLGLSNATVEWHIPDTAQPGIYRIRYFGH
NRKQDILKPAVILSFEGTSPAFEVVTI;
(ii)
DeLCify-2 (Length 653, Mass (Da) 72,006) having
the amino acid sequence below:

(SEQ ID NO: 7)
MPLFQNFSGYHIGVGRADCTGQVADINLMGYGKSGQNAQGILTRLYS
RAFIMAEPDGSNRTVFVSIDIGMVSQRLRLEVLNRLQSKYGSLYRRD
NVILSGTHTHSGPAGYFQYTVFVIASEGFSNQTFQHMVTGILKSIDIAH
TNMKPGKIFINKGNVDGVQINRSPYSYLQNPQSERARYSSNTDKEMI
VLKMVDLNGDDLGLISWFAIHPVSMNNSNHLVNSDNVGYASYLLEQ
EKNKGYLPGQGPFVAAFASSNLGDVSPNILGPRCINTGESCDNANSTC
PIGGPSMCIAKGPGQDMFDSTQIIGRAMYQRAKSKTCKPALGYSFAA
GTIDGVGGLNFTQGKTEGDPFWDTIRDQILGKPSEEIKECHKPKPILL
HTGELSKPHPWHPDIVDVQIITLGSLAITAIPGEFTTMSGRRLREAVQA
EFASHGMQNMTVVISGLCNVYTHYITTYEEYQAQRYEAASTIYGPHT
LSAYIQLFRNLAKAIATDTVANLSRGPEPPFFKQLIVPLIPSIVDRAPK
GRTFGDVLQPAKPEYRVGEVAEVIFVGANPKNSVQNQTHQTFLTVEKY
EATSTSWQIVCNDASWETRFYWHKGLLGLSNATVEWHIPDTAQPGIY
RIRYFGHNRKQDILKPAVILSFEGTSPAFEVVTI;
or
(iii)
DeLCify-3 (Length 643, Mass (Da) 70,961) having
the amino acid sequence below:
(SEQ ID NO: 8)
MPLFQNFSGYHIGVGRADCTGQVADINLMGYGKSGQNAQGILTRLYS
RAFIMAEPDGSNRTVFVSIDIGMVSQRLRLEVLNRLQSKYGSLYRRD
NVILSGTHTHSGPAGYFQYTVFVIASEGFSNQTFQHMVTGILKSIDIAH
TNMKPGKIFINKGNVDGVQINRSPYSYLQNPQSERARYSSNTDKEMI
VLKMVDLNGDDLGLISWFAIHPVSMNNSNHLVNSDNVGYASYLLEQ
EKNKGYLPGQGPFVAAFASSNLGDVSPNILGPRCINTGESCDNANSTC
PIGGPSMCIAKGPGQDMFDSTQIIGRAMYQRAKSKTCKPALGYSFAA
GTIDGVGGLNFTQGKTEGDPFWDTIRDQILGKPSEEIKECHKPKPILL
HTGELSKPHPWHPDIVDVQIITLGSLAITAIPGEFTTMSGRRLREAVQA
EFASHGMQNMTVVISGLCNVYTHYITTYEEYQAQRYEAASTIYGPHT
LSAYIQLFRNLAKAIATDTVANLSRGPEPPFFKQLIVPLIPSIVDRAPK
GRTFGDVLQPAKPEYRVGEVAEVIFVGANPKNSVQNQTHQTFLTVEKY
EATSTSWQIVCNDASWETRFYWHKGLLGLSNATVEWHIPDTAQPGIY
RIRYFGHNRKQDILKPAVILSFEG.

In another aspect, the present invention provides a pharmaceutical composition, comprising the polypeptide deLCify according to the invention, and a pharmaceutically acceptable carrier.

In one further aspect, the present invention provides a use of the polypeptide deLCify according to the invention for manufacturing a medicament for treatment of a cardiovascular disease.

In one yet aspect, the present invention provides a method for treating a cardiovascular disease in a subject comprising administering to the subject a therapeutically effective amount of the polypeptide deLCify according to the invention, or a pharmaceutical composition containing the polypeptide deLCify.

In the present invention, the LPCs comprise LPC (16:0), PC (18:0), LPC (20:0), LPC (22:0) and LPC (24:0).

In one example of the invention, the cardiovascular disease is an atherosclerotic cardiovascular disease.

The foregoing general description and the following detailed description are exemplary and explanatory only and are not restrictive of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings presenting the preferred embodiments of the present invention are aimed at explaining the present invention. It should be understood that the present invention is not limited to the preferred embodiments shown.

FIG. 1 shows the effects of deLCify-1 in degradation of excess LPC from lipid standard; wherein as compared to the control, 88% of LPC (16:0) was degraded (immunoprecipitation purified) and 82% of LPC was degraded (FPLC purified) (The p value was calculated using the two-tailed Student's t-test, and **** denoted p<0.0001).

FIG. 2 shows the effects of the deLCify-2 (LPCase-002), in which 92% of LPC (16:0) was degraded (immunoprecipitation purified), and 80% of LPC was degraded (FPLC purified) (The p value was calculated using the two-tailed Student's t-test, and **** denoted p<0.0001).

FIG. 3 shows the effects of the deLCify-3 (LPCase-003), in which 40% of LPC (16:0) was removed by 10 μg HP_deLCify-3, as compared to control. (The p value was calculated using the two-tailed Student's t-test, and **** denoted p<0.0001).

FIG. 4 shows the effects of the deLCify-1 (LPCase-001), in which 80% of LPC (16:0) was degraded (at the amount of 1 μg) and 92% of LPC was degraded (at the amount of 100 μg). (The p value was calculated using the two-tailed Student's t-test, and **** denoted p<0.0001).

FIG. 5A shows the efficacy of DeLCify-1 in degradation of excess LPC (16:0) in ASCVDs' LDL (n=3) (P value was determined by two-tailed Student's t-test, *** indicated p<0.001 (control group vs. disease group), ### indicated p<0.001 (disease group vs. treatment group).

FIG. 5B shows the efficacy of DeLCify-1 in degradation of excess LPC (18:0) in ASCVDs' LDL (n=3) (P value was determined by two-tailed Student's t-test, *** indicated p<0.001 (control group vs. disease group), ### indicated p<0.001 (disease group vs. treatment group).

FIG. 6 shows the area of collagen deposition quantified as Masson's trichrome-stained area as a percentage of the total area wherein the collagen deposition in the aorta was observed after treatment of a DeLCify-1, wherein the thoracic aortas of three groups of apoE^{-/-} mice (including the groups treated with saline, LPC and LPC plus DeLCify-1) were collected and stained with Masson's trichrome staining kit to assess the degree of collagen deposition in the aorta (n=3) shows the area of collagen deposition quantified as Masson's trichrome-stained area as a percentage of the total area (P value was determined by two-tailed Student's t-test, ** indicated p<0.01 (control group vs. disease group), ## indicated p<0.01 (disease group vs. treatment group)).

FIG. 7 shows the result of Masson's trichrome staining was obtained after treatment of a DeLCify-1, in which the collagen was stained in blue, in which the nuclei were stained in dark brown, muscle tissue was stained in red, and cytoplasm was stained in pink.

FIG. 8 shows that the LPC species were elevated especially in apoE^{-/-} injected with LPC, while DeLCify-1 attenuated the levels of LPC (n=5).

### DETAILED DESCRIPTION OF THE EMBODIMENTS

Unless otherwise defined herein, scientific and technical terms used herein have the meanings that are commonly understood by those of ordinary skill in the art.

As used herein, the abbreviations for each amino acid represented in three-letter code and one-letter code are given below.

TABLE 1
Three-letter and One-Letter Abbreviations of Each Amino Acid

| Amino acid | Three-letter Code | One-letter Code |
|---|---|---|
| Alanine | Ala | A |
| Arginine | Arg | R |
| Asparagine | Asn | N |
| Aspartic acid | Asp | D |
| Asn + Asp | Asx | B |
| Cysteine | Cys | C |
| Glutamine | Gln | Q |
| Glutamic acid | Glu | E |
| Gln + Glu | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein and in the appended claims, the singular forms “a,” “an,” and “the” include plural reference unless the context clearly indicates otherwise.

As used herein, “residue” refers to a position in a polypeptide or a protein and its associated amino acid identity.

As used herein, the term “functional fragment” refers to a fragment of a polypeptide that retains, for example, at least 10%, at least 20%, at least 30%, at least 40%, at least 50%, at least 60%, at least 70%, at least 80%, at least 90%, at least 95%, or 100% of at least one activity of the corresponding.

As used herein, the term “variant” refers to a variant of a peptide that retains, for example, at least 10%, at least 20%, at least 30%, at least 40%, at least 50%, at least 60%, at least 70%, at least 80%, at least 90%, at least 95%, or 100% of at least one activity of the corresponding polypeptide. For example, a variant can comprise a splice variant or a polypeptide comprising a mutation such as an insertion, deletion, or substitution.

“Percent (%) sequence identity” or “percent (%) identical to” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software. Those skilled in the art can determine appropriate parameters for aligning sequences, including any algorithms needed to achieve maximal alignment over the full length of the sequences being compared.

As used herein, the term “a functional fragment,” “variant” or “a modified polypeptide” of the polypeptide DeLCity refers to one or more polypeptide or functional fragment having the same or similar activity and function as the polypeptide DeLCity according to the invention, and includes equivalents thereof. Ranges of desired degrees of sequence identity are approximately 80% to 100% and intervening integer values. Typically, the percent identities between a disclosed sequence and a claimed sequence are 80% or more, 90% or more, or 95% or more.

As used herein, the phrase “substantially identical” refers to having a sequence identity that is 80% or more, for example 90% or more, e.g. 95%, 96%, 97%, 98%, 99%, 99.5%, 99.9%, or 100%, wherein the activity of the substantially identical polypeptide is unaltered by the modifications in the sequence that result in the difference in sequence identity.

The term “subject,” “patient” or “individual” is used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats), including dogs, cats, cattle, sheep, pigs, horses, rats, mice, guinea pigs, and etc. In some embodiments, the subject is a human.

As used herein, the terms “prevent,” “preventing” and “prevention” refer to the prevention of the recurrence or onset of, or a reduction in one or more symptoms of a disease or condition in a subject as result of the administration of a therapy (e.g., a prophylactic or therapeutic agent). For example, in the context of the administration of a therapy to a subject for an infection, “prevent,” “preventing” and “prevention” refer to the inhibition or a reduction in the development or onset of a disease or condition, or the prevention of the recurrence, onset, or development of one or more symptoms of a disease or condition, in a subject resulting from the administration of a therapy (e.g., a prophylactic or therapeutic agent), or the administration of a combination of therapies (e.g., a combination of prophylactic or therapeutic agents).

The term “treat,” “treating” or “treatment” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. Said carrier may be a diluent, a vehicle, an excipient, or a matrix to the polypeptide as an active ingredient.

The present invention provides a new polypeptide, called as “DeLCify,” comprising the amino acid sequence set forth in SEQ ID NO:1, or a functional fragment thereof, or a variant having an amino acid sequence.

In one embodiment of the invention, the polypeptide DeLCity has the amino acid sequence set forth in SEQ ID NO:1.

In addition, the polypeptide deLCify may have one of the additional fragments at N-terminus below:
an additional fragment of SEQ ID NO: 2; and
an additional fragment of SEQ ID NO: 3.

The polypeptide deLCify may have one of the additional fragments at C-terminus below:
an additional fragment of SEQ ID NO: 4; and
an additional fragment of SEQ ID NO: 5.

In one example of the invention, the functional fragment, variant or modified polypeptide thereof, is one having an amino acid sequence at least about 80%, at least about 85%, at least about 90%, at least about 95%, at least about 98%, at least about 99%, or at least about 99.5% identity thereto.

In the invention, the modified polypeptide of the polypeptide deLCify may include one or more substitutions below, wherein:
(1) the residue Alanine (A) may be substituted by Aspartic acid (D), Glutamic acid (E), Glycine (G), Serine (S), or Threonine (T);
(2) the residue Cysteine (C) may be substituted by Glycine (G), Arginine (R), Serine (S), Tryptophan (W) or Tyrosine (Y);
(3) the residue Aspartic acid (D) may be substituted by Alanine (A), Glutamic acid (E), Glycine (G), Histidine (H), Asparagine (N), Valine (V), Tyrosine (Y), Serine (S), or Threonine (T);
(4) the residue Glutamic acid (E) may be substituted by Alanine (A), Aspartic acid (D), Glycine (G), Lysine (K), Glutamine (Q), or Valine (V);
(5) the residue Glutamic acid (F) may be substituted by Isoleucine (I), Leucine (L) or Tyrosine (Y);
(6) the residue Glycine (G) may be substituted by Alanine (A), Cysteine (C), Aspartic acid (D), Glutamic acid (E) or Arginine (R);
(7) the residue Histidine (H) may be substituted by Aspartic acid (D), Leucine (L), Asparagine (N), Proline (P), Glutamine (Q), Arginine (R) or Tyrosine (Y);
(8) the residue Isoleucine (I) may be substituted by Glutamic acid (F), Leucine (L), Methionine (M), Asparagine (N) or Valine (V);
(9) the residue Lysine (K) may be substituted by Glutamic acid (E), Methionine (M), Asparagine (N), Glutamine (Q), Arginine (R) or Threonine (T);
(10) the residue Leucine (L) may be substituted by Glutamic acid (F), Histidine (H), Isoleucine (I), Methionine (M), Proline (P), Glutamine (Q), Arginine (R), Valine (V) or Tryptophan (W);
(11) the residue Methionine (M) may be substituted by Isoleucine (I), Lysine (K), Leucine (L), Arginine (R), Threonine (T) or Valine (V);
(12) the residue Asparagine (N) may be substituted by Aspartic acid (D), Histidine (H), Isoleucine (I), Lysine (K), Serine (S), Threonine (T) or Tyrosine (Y);
(13) the residue Proline (P) may be substituted by Histidine (H), Leucine (L), Glutamine (Q), Arginine (R) or Serine (S);
(14) the residue Glutamine (Q) may be substituted by Glutamic acid (E), Histidine (H), Lysine (K), Leucine (L), Proline (P) or Arginine (R);
(15) the residue Arginine (R) may be substituted by Cysteine (C), Glycine (G), Histidine (H), Lysine (K), Leucine (L), Methionine (M), Proline (P), Glutamine (Q), Threonine (T) or Tryptophan (W);
(16) the residue Serine (S) may be substituted by Alanine (A), Cysteine (C), Asparagine (N), Proline (P), Threonine (T), Tryptophan (W) or Tyrosine (Y);
(17) the residue Threonine (T) may be substituted by Alanine (A), Lysine (K), Methionine (M), Asparagine (N), Arginine (R) or Serine (S);
(18) the residue Valine (V) may be substituted by Aspartic acid (D), Glutamic acid (E), Isoleucine (I), Leucine (L) or Methionine (M);
(19) the residue Tryptophan (W) may be substituted by Cysteine (C), Leucine (L), Arginine (R) or Serine (S); and
(20) the residue Tyrosine (Y) may be substituted by Cysteine (C), Aspartic acid (D), Glutamic acid (F), Histidine (H), Asparagine (N), or Serine (S);
   wherein the above substitutions can be achieved according to the methods as disclosed by Pechmann & Frydman (Interplay between chaperones and protein disorder promotes the evolution of protein networks. PLOS Comput Biol. 2014 Jun. 26; 10(6):e1003674), the entire contents of which are incorporated by reference herein.

In the present invention, three specific examples include deLCify-1, deLCify-2 and deLCify-3, each of which has an unexpected efficacy in degradation of LPCs, including LPC (16:0), LPC (18:0), LPC (20:0), LPC (22:0) and LPC (24:0).

According to the invention, the polypeptide DeLCify was confirmed to provide an enzymatic activity that directly degrades lysophosphatidylcholine (LPC). In other words, the polypeptide DeLCify has lysophosphatidylcholine (LPC) toxicity-neutralizing activity, which can remove LPC in a subject. In addition, it was tested in animal models and found that the LPC was decreased after an injection of the polypeptide DeLCify.

Accordingly, the invention provides the polypeptide DeLCify with clinical application of treating diseases related to high levels of LPC, and provides a new method for the treatment of LPC-related disorders.

In addition, the present invention provides a method for treating a disease or disorder related to LPC (also known as “a LPC-related disorder”).

As used herein, the term “a disease or disorder related to LPC” or “a LPC-related disorder” refers to a disease or a disorder related to high levels of LPC. LPC is a major plasma lipid that has been recognized as an important cell signalling molecule produced under physiological conditions by the action of phospholipase A2 on phosphatidylcholine. LPC transports glycerophospholipid components such as fatty acids, phosphatidylglycerol and choline between tissues. LPC is also a major phospholipid component of oxidized low-density lipoproteins (Ox-LDL) and is implicated as a critical factor in the atherogenic activity of Ox-LDL. Hence, LPC plays an important role in atherosclerosis, and acute and chronic inflammation. It was found that increased levels of LPC in atherosclerosis, inflammatory disease, diabetes, adrenoleukodystrophy, and squamous cervical cancer, all of which are considered as examples of LPC-related diseases or disorders. In the present invention, the LPC-related diseases or disorders include but are not limited to a cardiovascular disease, a neurodegenerative disease, an inflammation-related disease, diabetes, and a cancer. A typical example of the LPC-related disease is atherosclerosis.

According to the present invention, the polypeptide DeLCify includes deLCify-1, deLCify-2, deLCify-3, and its variant, modified polypeptide or functional fragment having the same LPC toxicity-neutralizing activity and function as the DeLCify according to the invention.

The polypeptide DeLCify can be synthesized, recombinantly produced by a host cell, or isolated from a naturally occurring protein or organism, optionally with one or more modifications if necessary, using any standard or commonly used methodologies known to those skilled in the art.

The invention is further illustrated by the following example, which should not be construed as further limiting.

### Example

### Preparation of Plasmid DNA Construction for Expression of DeLCify

The pCMV6-Entry Mammalian Expression Vector was purchased from Origene (Origene Technologies; PS100001; USA). The full-length human-tagged open reading frame (ORF) clone of each of DeLCify-1, DeLCify-2 and DeLCify-3 was cloned to the vector by using PCR with the restriction enzymes SacI and NotI. First, the target insert DNAs were amplified with PCR and then the PCR products were isolated from the rest of the PCR reaction using the PCR Purification Kit. The PCR products were now ready for restriction digestion. Second, appropriate restriction enzymes were used to cleave both the plasmid vector and the insert target DNA of each DeLCify. Third, each digested plasmid vector was mixed and the DNA together with the DNA ligase enzyme was inserted, which catalyzed the formation of phosphodiester bonds. The ligase joined the cohesive ends of the insert and vector, resulting in a circular DNA molecule. The plasmid DNA was ready for transformation for selection, screening and verification.

### Transformation

The day before transformation, pure LB broth was prepared by mixing 25-gram LB broth base (Invitrogen, Cat #12780-052; USA) with 1-liter reverse osmosis (RO) water and LB agar plate was prepared by mixing 25-gram LB broth base (Invitrogen, Cat #12780-052; USA) with 1-liter RO water and 15-gram agar powder (Millipore, 05040; USA). All the ingredients were dissolved and mixed in a flask and sterilized by autoclave. After sterilization, the LB agar broth was cooled down to keep the temperature no more than 50° C., 50 μg/ml Kanamycin (ZEJU, K001; Taiwan) was added into the LB agar broth, and mixed well, then poured into 10-cm bacteria culture dishes, for 1 hour until agar solidification and the LB agar plates were stored in 4° C.

The pCMV6-containing plasmid DNA encoding for each of DeLCify-1, DeLCify-2 and DeLCify-3, was thawed with heat shock transformation and pipetted gently to mix the plasmid well, then placed on ice to prepare for transformation. The DH5-Alpha competent cells (ECOS™ 101, FYE678-10VL; Taiwan) were taken out from −80° C., directly added in 10 μL of the plasmid DNA (concentration ˜ 0.1 μg/μL), mixed well by vortex for 1-2 seconds, and then incubated on ice for 5 minutes. The water bath was preheated to reach 42° C. After 5 minutes of incubation, the competent cell mixture was heat shocked by transferring them to the preheated 42° C. water bath for 45 seconds, then incubated for 2 minutes on ice after heat-shock. 200 μL pure LB broth was added into competent cell mixture, incubated for 1 hour at 37° C., and shaken at 225 rpm. After 1 hour, the transformed cells were spread on Kanamycin selection LB plates, and incubated for 16˜18 hours in a 37° C. incubator.

### Colony PCR

Colony PCR is a simple and quick method for verifying the correct assembly of a cloned DNA construct (i.e., the pCMV6-containing plasmid DNA: (1) DeLCify-001; (2) DeLCify-002; or (3) DeLCify-003). First, the PCR mixture was prepared with T7 forward primer (Protech Technology; Taiwan), M13 reverse primer (Protech Technology; Taiwan), master mix (Thermo Fisher Scientific, K0171; USA), and UltraPure™ DNase/RNase-Free distilled water (Thermo Fisher Scientific, Cat #10977015; USA). The DNA samples were prepared with a 1.5 mL centrifuge tube. Each centrifuge tube was labelled and 20 μL pure LB broth was added into each tube. A sterile toothpick was used to pick the bacteria colony and mix it with 20 μL the pure LB broth. The PCR mixture and DNA sample were mixed in the PCR tube; the final volume in each tube was 18 μL of PCR mixture plus 2 μL of template DNA sample.

The reagents for colony PCR were listed in Table 2.

TABLE 2
Reagents for Colony PCR

| Reagent | Volume (per reaction) |
|---|---|
| PCR Master Mix (2X) | 10 μL |
| Forward Primer (10 μM) | 1 μL |
| Reverse Primer (10 μM) | 1 μL |
| Water | 6 μL |
| Template DNA | 2 μL |
| Total | 20 μL |

The conditions for colonies PCR were first denatured at 95° C. for 10 minutes, followed by 95° C. for 30 seconds, 52° C. for 40 seconds, 72° C. for 4 minutes as one cycle, this cycle was repeated for 30 times, and the last was annealing with 72° C. for 2.5 minutes and keeping in 25° C. This colony PCR was done by using the ProFlex™ PCR System of Applied Biosystem. The PCR products were stored at −20° C.

The conditions for colony PCR were listed in Table 3.

TABLE 3
Conditions for Colony PCR.

| Step | Temperature | Time | Cycles |
|---|---|---|---|
| Initial | 95° C. | 10 minutes | 1 |
| denaturation | | | |
| Denaturation | 95° C. | 30 seconds | 30 |
| Annealing | 52° C. | 40 seconds | |
| Extension | 72° C. | 2 minutes and 25 seconds | |
| | | (~1 kb/min) | |
| Hold | 25° C. | Indefinitely | |
| Initial | 95° C. | 10 minutes | 1 |
| denaturation | | | |

### Gel Electrophoresis

The colony PCR result was confirmed using 1% Agarose gel electrophoresis. First, agarose powder (AMRESCO, 0815-1006; USA) was mixed with 1×TBE buffer (AMRESCO, J885; USA), boil the agarose was boiled with microwave, and then cooled down the temperature no more than 50□C, then they were added in Ethidium Bromide (EtBr) nucleic acid dye (Yeastern Biotech, HYD007-A01; Taiwan), the flask was gently shaken, and the agarose gel mixed well with dye then all of them were poured into the pre-prepared gel electrophoresis system's container (Major Science, MT-105-S; Taiwan). Samples were loaded by mixing DNA with DNA loading dye (Bioman Scientific, DD01; Taiwan), and the marker we used is DNA KB Ladder Blue (HyCell, MB-1 KB; Taiwan). The gel was running for 40 minutes with 100V, then the results were detected with an imaging system (Vilber Lourmat Fusion Solo S; France). The bands should be located in the position between 2K and 3K.

### Plasmid DNA Extraction

The pCMV6-containing plasmid DNA for expression of (1) DeLCify-001, (2) DeLCify-002, or (3) DeLCify-003 was extracted by using Plasmid Midiprep Purification Kit (Genemark, DP01MD; Taiwan). Bacteria were added into a 500 mL flask with LB broth mixed with 50 μg/ml Kanamycin (ZEJU, K001; Taiwan), then cultured overnight in a 37° C. incubator with 200 rpm rotator speed. The day after, the bacteria pellet was harvested by transferring all the liquid bacteria into a 50 mL centrifuge tube, centrifugation for 5 minutes at 12,000˜14,000 g with an ultracentrifuge (Beckman Coulter, Optima L-100K; USA). The supernatant was discarded and any excess media was removed. After that, the bacteria pellet was completely resuspended in 5 mL Solution I by pipetting or vortex. It was incubated at room temperature for 10 minutes, to obtain a uniform and complete suspension of cells, especially for high density (>3×10⁹ cells/mL) culture or large volume (>250 mL) culture. Five mL Solution II was added and mixed by inverting the tube 6˜8 times; the cell suspension should turn clear immediately. It was incubated again at room temperature for 2˜4 minutes. Five mL Solution III was added and mixed by inverting the tube 6˜8 times. It was incubated on ice for 10 minutes. The lysate was centrifuged at 12,000˜14,000 g for 10 minutes at 4° C. A compact white pellet was formed along the side or at the bottom of the tube. All the supernatant was collected into a new 50 mL centrifuge tube and added in 10 mL isopropanol (Sigma-Aldrich, #19516; Germany), mixed by inverting 6˜8 times and centrifuged at 4° C., 12,000˜14,000 g for 10 minutes. All the supernatant after centrifuge was discarded and the DNA pellet was resuspended with 500 μL Binding Solution, and mixed well by pipetting gently. The Spin Column was inserted into a Collection Tube, all the solution was carefully transferred from the step above into the spin column, equilibration for 1 minute, and then centrifuged at top speed (12,000˜14,000 g) for 2 minutes with a microcentrifuge. The filtrate was discarded from the collection tube, 500 μL of the binding solution was added to the spin column, and equilibrated for 2 minutes, and then centrifuged at top speed (12,000˜14,000 g) for 2 minutes. This step was repeated for two more times. The filtrate was discarded from the collection tube and 650 μL of Endotoxin Removal Wash Solution was added to the spin column, equilibrated for 2 minutes, then centrifuged at top speed (12,000˜14,000 g) for 2 minutes. This step was repeated for two more times. The filtrate was discarded from the collection tube, 600 μL of wash solution was added to the spin column then centrifuged at top speed (12,000˜14,000 g) for 2 minutes. This step was repeated for four more times. The filtrate was discarded and centrifuged at top speed for additional 5 minutes and the spin column was put in a 60° C. incubator for 10 minutes to remove residual traces or ethanol. The spin column was transferred into a new 1.5 mL centrifuge tube and 100 μL of preheated (60° C.) Elution solution or sterile ddH₂O was added into the centrifuge tube, equilibration for 3 minutes and the last step was to centrifuge it at top speed for 2 minutes to elute the plasmid DNA. The concentration and purity were checked after work. The DNA was quantified by a microplate spectrophotometer (BioTek, Epoch; USA). The yield of plasmid DNA should be at least 1000 ng/μL for 300 mL E. coli culture at a purity of 1.8-2.0 (A260/280). The extracted plasmid DNA was stored at −20° C.

### Cell Culture

To overproduction of target protein, the Human Embryonic Kidney Cells 293 (HEK 293-T; mutant version of the SV40 large T antigen) were maintained in a T-75 culture flask with 10 mL 1× Dulbecco's Modified Eagle Medium (DMEM) (Gibco Life Technologies, 11885-076; Canada) which included 10% of Fetal Bovine Serum (FBS) (Gibco Life Technologies, 10437-028; Canada) and 1% of Penicillin-Streptomycin-Ampho.B (P/S/A) (Biological Industries, 03-033-1B, USA). For subculture, the culture medium was discarded and the monolayers were gently rinsed three times with 10 mL 1× sterile Phosphate-buffered Saline (PBS) (Genemark, GB07-1-S; Taiwan). One mL of 1× Trypsin-EDTA solution (Gibco Life Technologies, 15400-054; Canada) was slowly added to cover the cell monolayer. It was incubated at 37° C. for 5 minutes to split and disrupt the cell monolayers. The cells should not be agitated by hitting or shaking the flask while waiting for the cells to detach. After 5 minutes, 9 mL of DMEM was added into the T-75 culture flask to stop the reaction activity of trypsin-EDTA and the cells were suspended by gently pipetting. All the cells' liquid was transferred to a 15 mL centrifuge tube and centrifuged at 1500 rpm for 5 minutes. The medium containing trypsin-EDTA was discarded and the cells were re-suspended with fresh DMEM by pipetting gently, 1×10⁶ cells were seeded into a new T-75 culture flask, and DMEM was added to reach 10 mL in the final volume.

### Transfection and Confirmation

Transfection was done by using Lipofectamine 3000 (Invitrogen, L3000-015, USA) reagent. The day before transfection, seeding HEK 293-T with a density of approximately 2.2×10⁶ cells, the cells should be 70˜90% confluent at the time of transfection. The next day, two 15 mL centrifuge tubes were prepared and marked as tubes A and B. First, in tube A, 26 μL Lipofectamine™ 3000 reagent was diluted in 1500 μL Opti-MEM™ medium, and then mixed well by inverting for 4˜5 times, incubated at room temperature for 10 minutes. After that, the master mix of DNA was mixed well with tube B by diluting 15 μg DNA in 1500 μL OptiMEM™ medium, then 30 μL of P3000™ reagent was added into the mixture and mixed well by inverting again. The diluted DNA mixture was added to Tube B, containing a diluted Lipofectamine™ 3000 reagent, and incubated for 10 min at room temperature. Add DNA-lipid complex to cells, and incubated at 37° C. for 5 hours. After 5 hours, 4 mg/mL G418 (ZEJU, G001, Taiwan) antibiotic was added and incubated at 37° C. for 48 hours. After 48 hours, the transfected cells were visualized and analyzed by using an imaging system (EVOS® FL Imaging System, Thermo Fisher Scientific; USA).

### Harvest Protein

The culture medium was removed and the cell monolayers were washed two times with 10 mL 1× sterile Phosphate-buffered Saline (PBS) (Genemark, GB07-1-S; Taiwan). The cell lysis buffer was prepared by mixing RIPA lysis buffer (VWR Life Science, VWRVN653; USA) with 1× Complete EDTA free Protease Inhibitor (ROCHE, cat #05056489001; Switzerland). One mL cell lysis buffer was added to the T-75 culture flask and incubated for 5 minutes on ice. All the cells were scraped down, collected in a new 1.5 mL centrifuge tube, and sonicated for 10 minutes in a water bath with ice. The cells were centrifuged at 15,000 rpm for 15 minutes to remove the cell debris, the supernatant was collected to a new centrifuge tube, and then for protein quantification. After quantification, the protein was stored at −20° C. The protein was quantitative using Pierce™ BCA Protein Assay Kit (Invitrogen, cat #23225; USA), and the result was read by microplate spectrophotometer (BioTek, Epoch; USA).

### Generation of Stable Cell Lines

The transfected cells were detached with 1× Trypsin-EDTA (Gibco, ref. 15400-054; Canada), the cells were split down, and washed with 1× sterile Phosphate-buffered Saline (PBS) (Genemark, GB07-1-S; Taiwan) for three times. After that, the cell pellets were resuspended with 1 mL 1×PBS, containing 10% of Fetal Bovine Serum (FBS) (Gibco, ref. 10437-028, USA) and 4 mg/mL G418 (ZEJU, G001; Taiwan) for fluorescence-activated cell sorting (FACS). After FACS with GFP positive selection, a single cell was sorted into each well in a 96-well plate. The single cell was cultured in 1×DMEM with 4 mg/mL G418 (ZEJU, G001; Taiwan), and the media was replaced with the selected antibiotic every 2˜3 days for up to a week. The single cell was expanded to a T-175 culture flask for cell banking and protein overproduction.

Protein Purification by FPLC Equipped with Affinity his-Trap Column

First, the buffers A and B were prepared for affinity purification. Buffer A was an equilibration buffer including 50 mM Tris Base (Avantor, J.T.Baker 4109-06, New Jersey; USA) and 300 mM Sodium Chloride (NaCl) (Honeywell, cat #31434; Germany) with pH 8.0; and buffer B was an elution buffer including 50 mM Tris Base (Avantor, J.T.Baker 4109-06, New Jersey; USA), 300 mM Sodium Chloride (NaCl) (Honeywell, cat #31434; Germany) and 250 mM Imidazole (Sigma-Aldrich, cat #12399; Germany) with pH 8.0. The column is Bio-Scale™ Mini Nuvia™ IMAC Ni-Charged, Cartridges 1 mL/5 mL (Bio-Rad, California; USA), and the system was fast column liquid chromatography (FPLC) from Bio-Rad (NGC Chromatography, California; USA).

The protein samples were prepared and the volume was concentrated to become no more than 2 mL. The 2 mL sample was injected into the FPLC system, the unbinding protein was washed away with equilibration buffer, and the non-specific binding protein was washed away with 1% elution buffer and the target protein was eluted with 50% elution buffer. The system was running with 0.5 ml/min for 1 mL column and 1.5 ml/min for 5 mL column. The methods for His-trap purification were first equilibration for 10 CVs (column volume; units), then the sample was injected into the system after equilibration. Next, the unbinding protein was washed away with buffer A for 10 CVs, and the unspecific binding protein was washed out with 1% buffer B for 10 CVs. The targeted protein was eluted with 50% of buffer B for 5 CVs, and the column was washed with 100% of buffer B for 5 CVs. Last, the column and the whole system were washed with buffer A for another 10 CVs to remove the remaining Imidazole. The eluted fraction was collected, following by a buffer exchange.

### Buffer Exchange, Concentration and Quantification

All the fractions were collected separately and continued with buffer exchange to remove the excess Imidazole. Buffer A was used as a storage buffer for targeted proteins. The buffer exchange was done by using PD-10 Desalting Columns (GE Healthcare; USA), and the purified protein was concentrated with 50 kilodaltons (kDa) cut-off Regenerated Cellulose (RC) Membrane Filter (Sartorius; Germany). Ultimately, the protein concentration was quantified by Pierce™ BCA Protein Assay Kit (Thermo Fisher Science, Prod #23225; USA). The results were confirmed by sodium dodecyl sulphate-polyacrylamide gel electrophoresis (SDS-PAGE). Signals were archived using Immobilon Western Chemiluminescent HRP Substrate (Millipore; USA) and then visualized and recorded with the Fusion Solo S imaging system (Vilber Lourmat; France).

### Protein Purification by FPLC-Size Exclusion Column

The size exclusion purification was performed after His-trap purification to collect the most purified protein. The target protein was separated according to the standard purchased from Bio-Rad (Bio-Rad, Gel Filtration Standard #1511901; USA). The protein standard was isolated into six groups according to their molecular weight (kDa), from the largest to the smallest, Protein aggregates (void peak), Thyroglobulin (670 kDa), γ-globulin (158 kDa), Ovalbumin (44 kDa), Myoglobin (17 kDa) and Vitamin B12 (1.35 kDa). Buffer A in His-trap purification was used as a mobile phase, and the column for separation was Superose 6 10/300 GL size exclusion column (GE Healthcare Life Science, #17517201; USA). The Superose 6 10/300 GL was used as a pre-packed column for high-performance size exclusion chromatography separation and analysis within the broad fractionation range for molecules between 5,000 kDa to 5,000,000 kDa. The purification was performed with Bio-Rad NGC Chromatography (California; USA). The condition for SEC purification was 100% buffer A with a flow rate of 0.5 ml/min, and the sample volume load per round was 500 μL. After SEC, the targeted protein was collected and followed with concentration and quantification. The results were confirmed by using SDS-PAGE and western blot analysis.

### SDS-PAGE and Western Blot Confirmation

4% of stacking gel mixture and 10% of separating gel mixture including 1.5 M Tris HCl pH 8.8 (Biomate, BR270; Taiwan), 0.5 M Tris HCl pH 6.8 (Biomate, BR210; Taiwan), 10% Sodium Dodecyl Sulfate (SDS) (VWR Life Science, 0227; USA), 30% Arcylamide (Bio-Rad, #1610156; USA), Tetramethylethylenediamine (TEMED) (Sigma-Aldrich, T9281; USA), 10% Ammonium Persulfate (APS) (Sigma-Aldrich, A3678; USA) were prepared. All the ingredients were mixed by stirring for 20 seconds, then poured into the rack, the suitable comb was put, and waiting until the gel became solidification.

The gel was running with Bio-Rad western blot system at 70 V for 45 minutes for stacking gel, and changed to 110 V for 90 minutes for separating gel. SDS-PAGE was stained using Simply Blue™ Safe Stain (Invitrogen, LC6060-465043; USA) overnight in a 4° C. rotator. For western blot, the protein was directly transferred to a PVDF membrane (Millipore Immobilon-P Transfer Membranes, IPVH 00010; Germany). The condition for transfer was 170 mA, running for 1 hour on ice. The membrane was blocked with 10% milk (Fonterra Anchor Milk Powder; New Zealand) in TBST buffer for at least 30 minutes and incubated with primary antibody for 1 hour under room temperature. Primary antibodies were DeLCify, His Tag Antibody (GeneTex, GTX 115045; USA), Monoclonal anti-β-actin Antibody (Sigma-Aldrich, A5441; USA) and secondary antibodies are Mouse IgG (HRP) antibody (GeneTex, GTX 213112-25; USA) and Rabbit IgG (HRP) antibody (GeneTex, GTX 213110; USA). Signals were archived using Immobilon Western Chemiluminescent HRP Substrate (Millipore; USA) and then visualized and recorded with the Fusion Solo S imaging system (Vilber Lourmat; France).

### Enzyme Function Test

The purified enzyme (1) DeLCify-001; (2) DeLCify-002; (3) DeLCify-003 were incubated with wither LDL samples or lysophosphatidylcholines (LPC) lipid standard (C16:0 LysoPC, Avanti, #855675; USA) under 37° C. with 125 revolutions per minute (rpm) shaking condition for 2 hours. The enzyme buffer was prepared for the enzyme function test. The enzyme buffer was constituted with 25 mM Tris Hydrochloride (Tris-HCL) (Avantor, J.T.Baker 4103-02, New Jersey, USA) pH 7.4, 2.5 mM Magnesium Chloride (MgCl2) anhydrous (Sigma Life Science, M8266, USA), 2.5 mM Calcium Chloride (CaCl2) anhydrous (Shimakyu's Pure Chemical, Japan), 150 mM Sodium Chloride (NaCl) (Honeywell, cat #31434-5 KG, Germany).

The conditions for enzyme function test with LDL were given in Table 4.

TABLE 4
Condition for Enzyme Function Test with LDL:

| Enzyme function test | LDL (Control) | LDL + DeLCify (Test) |
|---|---|---|
| Enzyme (μL) | x | 30 μg ^{a} |
| (1) DeLCify-001; | | |
| (2) DeLCify-002; | | |
| (3) DeLCify-003 | | |
| LDL (μg) | 100 μg ^{b} | 100 μg ^{b} |
| ddH₂O (μL) | 180 − volume of | 160 − volume of |
| | LDL ^{c} | LDL − volume of |
| | | LPCase ^{d} |
| 10x enzyme buffer pH 7.4 | 20 | 20 |
| (μL) | | |
| Total volume (μL) | 200 | 200 |

^{a} For DeLCify with concentration 0.5 μg/μL, 30 μg should be 60 μL.
^{b} For LDL with concentration 2.0 μg/μL, 100 μg should be 50 μL.
^{c} The volume for ddH₂O in control will be 180 − 50 = 130 (μL).
^{d} The volume for ddH₂O in test will be 180 − 50 − 60 = 70 (μL).

The condition for enzyme function test with lipid standard was given in Table 5.

TABLE 5
Condition for Enzyme Function Test with Lipid Standard:

| Enzyme function test | STD (Control) | STD + LPCase (Test) |
|---|---|---|
| Enzyme (μL) | X | 5 μg ^{a} |
| (1) DeLCify-001; | | |
| (2) DeLCify-002; | | |
| (3) DeLCify-003 | | |
| C16:0 LysoPC (μM) | 10 | 10 |
| Lipids Solution (μL) | 200 | 200 |
| ddH₂O (μL) | 160 | 160 − volume of |
| | | LPCase ^{b} |
| 10x enzyme buffer pH 7.4 (μL) | 40 | 40 |
| Total volume (μL) | 400 | 400 |

^{a} For DeLCify with concentration 0.5 μg/μL, 5 μg should be 10 μL.
^{b} The volume for ddH₂O will be 160 − 10 = 150 (μL).

### Lipids Extraction

The lipids were extracted with the Folch method. In brief, samples were diluted/mixed with MS grade water (PURELAB Option-Q, UK) to reach 0.8 mL in final, followed by adding 2 mL methanol (A456-4, LC/MS Grade, Thermo Fisher Scientific, Waltham, MA, USA) and 1 mL chloroform (9831-2, LC/MS reagent, J.T.Baker, Phillipsburg, NY, USA) in the ratio of 2:1:0.8 (methanol:chloroform:sample). The mixture was vortexed for 15 seconds. Then, 1 mL of MS-grade water and 1 mL of chloroform was added, and the mixture was vortexed. The sample tubes were centrifuged at 800 RCF, 4° C., for 10 minutes. The upper aqueous solvent was discarded, and the lower organic phase was collected into a new glass vial, which was then evaporated with a nitrogen gas evaporator (Eyela MG-2200; Japan). It was then solubilized using lipid buffer (2:1:1; Isopropanol, LC/MS Grade Merck Millipore, MA, USA; Acetonitrile; H₂O, LC/MS reagent, 9831-2, J.T.Baker, Phillipsburg, NY, USA) and stored at −80° C. until LC/MS^{E} analysis.

### LC/MS^{E} Analysis

When the samples were ready for LC/MS^{E} analysis, dissolve the samples with 200 μL of lipid solution. To prepare the lipid solution, Isopropanol (Fisher Chemical, A461-4; Canada), Acetonitrile (Fisher Chemical, A955-4; Canada), and mass grade water were mixed according to the ratio of 2:1:1. The lipid solution was added to the sample, and mixed well by vortex for at least 10 seconds. Finally, the samples were ready for ultra-performance liquid chromatography (UPLC)/MS^{E} to analyze the composition of lipids.

The chromatography analysis was performed by ACQUITY UPLC® CSH™ C18 2.1×100 mm, 1.7 μm as the column for UPLC, at the column temperature of 55□C, the flow rate of 0.1 ml/min. for 20 minutes. Buffer A2 was stand as the mobile phase solution and the ingredients were acetonitrile/water (60:40) mixed with 10 mM ammonium formate and 0.1% formic acid. The components of mobile phase B2 were Isopropanol (Fisher Chemical, A461-4; Canada)/Acetonitrile (Fisher Chemical, A955-4; Canada) (90:1) mixed with 10 mM ammonium formate and 0.1% formic acid. The conditions of UPLC in lipids isolation were given in Table 6.

TABLE 6
The Condition of UPLC in Lipids Isolation.

| Time(min) | Flow (mL/min) | % A^{a} | % B^{b} | Curve^{c} |
|---|---|---|---|---|
| Initial | 0.1 | 60 | 40 | Initial |
| 2.0 | 0.1 | 57 | 43 | 6 |
| 2.1 | 0.1 | 50 | 50 | 1 |
| 12.0 | 0.1 | 46 | 54 | 6 |
| 12.1 | 0.1 | 30 | 70 | 1 |
| 18.0 | 0.1 | 1 | 99 | 6 |
| 18.1 | 0.1 | 60 | 40 | 6 |
| 20.0 | 0.1 | 60 | 40 | 1 |

^{a}Acetonitrile/water (60:40); 10 mM ammonium formate; 0.1% formic acid.
^{b}Isopropanol/acetonitrile (90:10); 10 mM ammonium formate; 0.1% formic acid.
^{c}Curve 1 stands for step change; curve 6 stands for linear change.

As shown in Table 5, the flow rate was 0.1 ml/min, and the gradient of buffer was 60% A2 with 40% B2 at the beginning; at 2 minutes, the gradient of buffer was 57% A2 with 43% B2; at 2.1 minutes, gradient of buffer was 50% A2 with 50% B2; at 12 minutes, gradient of buffer was 46% A2 with 54% B2; at 12.1 minutes, gradient of buffer was 30% A2 with 70% B2; at 18 minutes, gradient of buffer was 1% A2 with 99% B2; at 18.1 minutes, gradient of buffer was 60% A2 with 40% B2; at 20 minutes, gradient of buffer was 60% A2 with 40% B2.

In this experiment, the Xevo G2 QTOF/UPLC (Waters; USA) mass spectrometry was used. The samples were ionized from the liquid phase by; samples passed through the LC column by Electrospray Ionization (ESI), the fluid flowed through the tubing, and then reached the probe. There was a stainless-steel capillary in the middle of the probe. At the same time, a high voltage (positive charge: 2.0 KV; negative charge: 1.0 KV) brought the ionized samples directly into the mass spectrometry, and the exact m/z of the samples were detected (cone voltage 30 kV; Collision energy 35˜55V). The signals were detected between 250˜1600 Da. at the voltage of 35˜55 V, every 10 seconds, the leucine enkephalin (556.2771 Da) (Waters, 700002456; USA) did real-time calibration, cone voltage 2.65 kV, collision energy 0˜6 V. The total duration for the whole process was 20 minutes, and data were analyzed and quantified by Progenesis QI (Waters, Nonlinear Dynamics; USA) statistic software.

### Animal Study

The six-week-old apoE^{-/-} mice (C57BL/6-Apoe^{em1Narl}/Narl), purchased from National Applied Research Laboratories Taiwan, were kept under SPF conditions. After acclimation until eight-week-old, mice were randomly divided into different dietary or treatment groups. To evaluate the therapeutic functions of the DeLCifys, LPC was injected into normal chow diet (NCD)-fed apoE^{-/-} mice to promote atherosclerosis (LPC group); and co-treated with DeLCify to examine therapeutic effects (LPC+DeLCify group). ApoE^{-/-} mice fed with NCD served as control (Control group). The NCD fed was purchased from Research Diets Inc. (D12450B; USA), which included 10 kcal % fat. The LPC standard was dried and redissolved in 1% of bovine serum albumin (BSA) as the carrier, then diluted with normal saline to reach the final concentration at 200 μM. Normal saline was also used to buffer the DeLCifys. For the different interventions, either 200 μM LPC or 3 mg/kg DeLCify was injected into apoE^{-/-} mice through the tail vein thrice a week. At the end of the study (16-week-old), the mice were sacrificed. The mice plasma was collected to examine the lipid alteration, particularly the LPC and ceramide, by using mass spectrometry. The aorta was stained with Hematoxylin and Eosin (H&E), Masson's trichrome to examine the collagen deposition which is the sigh of vascular fibrosis, and Verhoeff-Van Gieson (VVG) staining to examine the elastic fiber.

### Histopathology and Histochemistry Staining (Masson's Trichrome)

The Masson's Trichrome staining kit was purchased from Sigma-Aldrich (HT15; USA). The mice's aortic samples were embedded in a paraffin block and cut into 5 μm thick cross-sections. Aortic sections were deparaffinized and stained with Masson's trichrome. In brief, the slides were deparaffinized and hydrated in deionized water. The slides were stained in preheated Bouin's Solution at 56° C. for 15 minutes, then the slides were cooled in tap water (18˜26° C.) and held in a Coplin jar. The slides were washed under running water to remove the yellow tint from the portions. After that, the slides were stained for 5 minutes in Working Weigert's Iron Hematoxylin Solution and washed for 5 minutes in running tap water to remove any excess stain. After that, the slides were stained for 5 minutes in Biebrich Scarlet-Acid Fucshin and rinsed in deionized water. The slides were immersed in Working Phosphotungstic/Phosphomolybdic Acid Solution for 5 minutes. The slides should then be submerged in Aniline Blue Solution for 5 minutes. Finally, the slides were dip in 1% Acetic Acid for 2 minutes before discarding the solution and rinsing the slides. Ultimately, the slides were dehydrated in alcohol, clarified in xylene, and mounted. The results were captured and analyzed by TissueFAXS (TissueGnostics; Austria), and quantified by ImageJ (National Institutes of Health; Maryland; USA).

### Results

### 1. LPC can be Removed by DeLCify In Vitro.

To test the enzyme purity and function, the crude extract of DeLCify was taken from the pre-established DeLCify-expressing HEK 293T cells and purified by immunoprecipitation (IP_deLCify-1) or FPLC (HP_deLCify-1) equipped with a Ni-charged affinity column. For the enzymatic function, 30 μg IP_deLCify-1 or HP_deLCify-1 were incubated with 100 μM LPC(16:0), respectively. As compared to the control, 30 μg IP_deLCify-1 can eliminate 88% of LPC (p<0.0001), and 30 μg HP_deLCify-1 can remove 82% of LPC (p<0.0001), see FIG. 1.

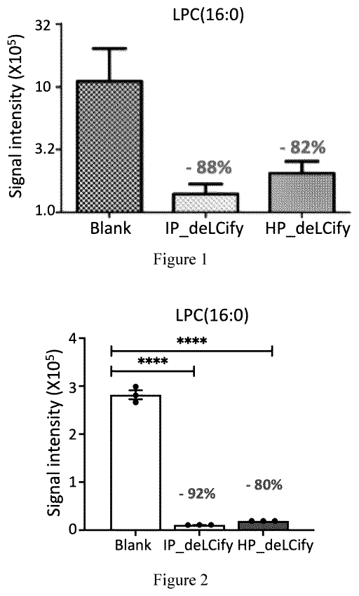

To test the function of shortened form of enzyme, the crude extract of deLCify-2 was taken from the pre-established deLCify-expressing HEK 293T cells and purified by immunoprecipitation (IP_deLCify-2) or FPLC (HP_deLCify-2) equipped with a Ni-charged affinity column. For the enzymatic function, 10 μg IP_deLCify-2 or HP_deLCify-2 were incubated with 30 μM LPC(16:0), respectively. As compared to the control, 10 μg IP_deLCify-2 can eliminate 92% of LPC (p<0.0001), and 10 μg HP_deLCify-2 can remove 80% of LPC (p<0.0001), see FIG. 2.

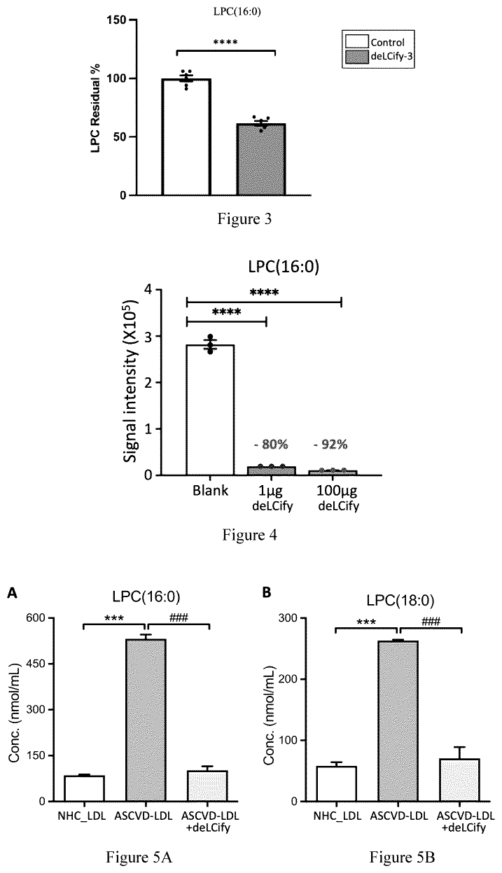

To test the function of shortened form of enzyme, the crude extract of deLCify-3 was taken from the pre-established deLCify-expressing HEK 293T cells and purified by FPLC (HP_deLCify-3) equipped with a Ni-charged affinity column. For the enzymatic function, 10 μg HP_deLCify-3 were incubated with 30 μM LPC(16:0. As compared to the control, 10 μg HP_deLCify-3 can remove 40% of LPC (p<0.0001), see FIG. 3.

To test the range of enzyme function, the crude extract of deLCify-1 was taken from the pre-established deLCify-expressing HEK 293T cells and purified by FPLC (HP_deLCify-1) equipped with a Ni-charged affinity column. 1 μg or 100 μg HP_deLCify-1 was incubated with 30 μM LPC(16:0), respectively. As compared to the control, 1 μg HP_deLCify-1 can eliminate 80% of LPC (p<0.0001), and 100 μg HP_deLCify-1 can remove 92% of LPC (p<0.0001), see FIG. 4.

2. The Excess LPC in ASCVDs' LDL could be Removed by DeLCify in Vitro.

LDLs were isolated from the age- and gender-matched normal healthy controls (NHCs) and ASCVD patients (ASCVDs). By lipid extraction and mass spectrometry analysis, the dominant LPC in humans' LDL, including LPC(16:0) and LPC(18:0) were detected. After the addition of DeLCify to ASCVDs' LDL respectively following with a two-hour incubation under 37° C., mixing and shaking condition. The enzymatic functions were confirmed and analyzed by mass spectrometry. The results were given in FIG. 5A and FIG. 5B, showing that the DeLCify could remove the excess LPC from the patients' LDL, including LPC (16:0) and LPC (18:0) respectively, the elevated concentration in ASCVDs could be decreased by 30 μg by DeLCify-1. As shown in FIG. 5A, the results indicated that DeLCify-1 could decrease the concentration of LPC (16:0) from 532 nM to 102 nM (^{###}p<0.001). As shown in FIG. 5B, the DeLCify-1 could decrease the concentration of LPC (18:0) from 263.3 nM to 70.7 nM (^{###}p<0.001). Taken together, the concentration of LPC were 4.5 times higher found in ASCVDs' LDL as compared to NHCs; however, the excess LPC in ASCVDs LDL could be removed and brought back to normal ranges after treatment with DeLCify.

3. The Collagen Deposition in Thoracic Aortas of LPC-Injected apoE^{-/-} Mice was Attenuated after the Second Injection of DeLCify.

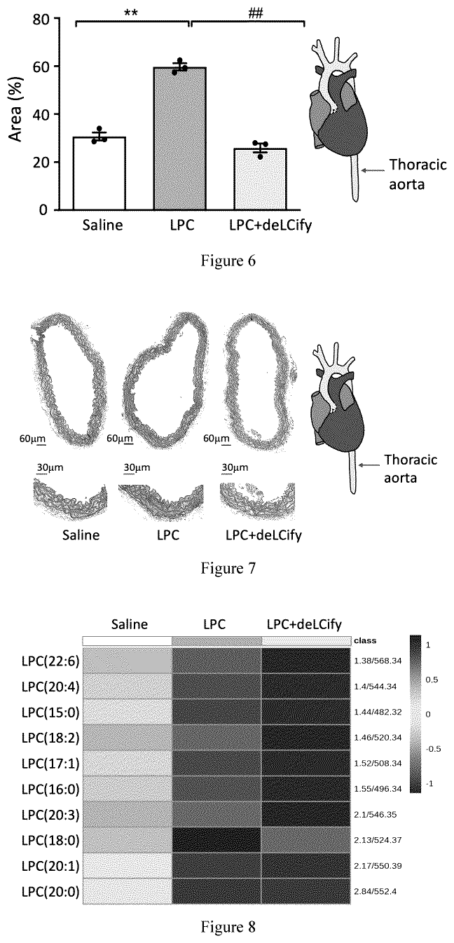

Collagen fibers are the main component of atherosclerotic lesions. The thoracic aortas of three groups of apoE^{-/-} mice were collected and stained with Masson's trichrome staining kit to assess the degree of collagen deposition in the aorta. The area of collagen deposition was typically quantified as Masson's trichrome-stained area as a percentage of the total area. As shown in FIG. 6, the highest percentage of the stained collagen was found in apoE^{-/-} mice injected with LPC, and they showed attenuation after the second injection of DeLCify. Data were quantified as shown in FIG. 7.

4. The Levels of LPC were Attenuated in apoE^{-/-} Injected with DeLCify.

The mouse plasma was taken for lipid extraction and identification. It was discovered that LPC species with different branches were higher in the LPC-injected mice than those in the saline-injected mice. As shown in FIG. 8, all LPC species were significantly decreased after the injection of DeLCify to pre-LPC-injected mice.

In view of the above, the DeLCifys according to the invention are effective in degradation of LPCs, including 16:0, 18:0, 20:0, 22:0 and 24:0, which should be potential to be developed as a drug for degrading LPCs in a patient.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

What is claimed is:

1. A polypeptide DeLCify with lysophosphatidylcholine (LPC)-degrading activity, which comprises the amino acid sequence set forth in SEQ ID NO: 1, a variant, a modified polypeptide or a functional fragment thereof.

2. The polypeptide DeLCify of claim 1, wherein the LPC includes comprises LPC(16:0), PC(18:0), LPC(20:0), LPC(22:0) or LPC(24:0).

3. The polypeptide DeLCify of claim 1, wherein the polypeptide DeLCify is effective in treatment of a LPC-related disease or disorder.

4. The polypeptide DeLCify of claim 1, wherein the LPC-related disease or disorder is a cardiovascular disease or a neurodegenerative disease.

5. The polypeptide DeLCify of claim 1, wherein the LPC-related disease or disorder is acute or chronic inflammation.

6. The polypeptide DeLCify of claim 1, wherein the LPC-related disease or disorder is an inflammatory disease.

7. The polypeptide DeLCify of claim 1, wherein the LPC-related disease or disorder is diabetes.

8. The polypeptide DeLCify of claim 4, wherein the cardiovascular disease is an atherosclerotic cardiovascular disease.

9. The polypeptide DeLCify of claim 4, wherein the cardiovascular disease is atherosclerosis.

10. The polypeptide DeLCify of claim 1, wherein the polypeptide DeLCify has a sequence identity of 80% or more to SEQ ID NO:1.

11. The polypeptide DeLCify of claim 1, wherein the polypeptide DeLCify has a sequence identity of 90% or more to SEQ ID NO:1.

12. The polypeptide DeLCify of claim 1, wherein the polypeptide DeLCify has a sequence identity of 95% or more to SEQ ID NO:1.

13. The polypeptide DeLCify of claim 1, wherein the polypeptide deLCify further comprises an additional fragment at N-terminus selected from the group consisting of the amino acid sequences set forth in SEQ ID NO: 2 and SEQ ID NO: 3.

14. The polypeptide DeLCify of claim 1, wherein the polypeptide deLCify further comprises an additional fragment at C-terminus selected from the group consisting of the amino acid sequences set forth in SEQ ID NO: 4, the sequence having a sequence identity of 80% or more to SEQ ID NO:4, SEQ ID NO: 5, and the sequence having a sequence identity of 80% or more to SEQ ID NO:5.

15. The polypeptide DeLCify of claim 1, wherein the polypeptide deLCify comprises the amino acid sequence set forth in SEQ ID NO:1, and the amino acid sequences set forth in SEQ ID NO: 4 at C-terminus.

16. The polypeptide DeLCify of claim 1, wherein the polypeptide deLCify comprises the amino acid sequence set forth in SEQ ID NO:1, and the amino acid sequences set forth in SEQ ID NO: 5 at C-terminus.

17. The polypeptide DeLCify of claim 1, wherein the modified polypeptide includes one or more substitutions in which:
(1) the residue Alanine (A) may be substituted by Aspartic acid (D), Glutamic acid (E), Glycine (G), Serine (S), or Threonine (T);
(2) the residue Cysteine (C) may be substituted by Glycine (G), Arginine (R), Serine (S), Tryptophan (W) or Tyrosine (Y);
(3) the residue Aspartic acid (D) may be substituted by Alanine (A), Glutamic acid (E), Glycine (G), Histidine (H), Asparagine (N), Valine (V), Tyrosine (Y), Serine (S), or Threonine (T);
(4) the residue Glutamic acid (E) may be substituted by Alanine (A), Aspartic acid (D), Glycine (G), Lysine (K), Glutamine (Q), or Valine (V);
(5) the residue Glutamic acid (F) may be substituted by Isoleucine (I), Leucine (L) or Tyrosine (Y);
(6) the residue Glycine (G) may be substituted by Alanine (A), Cysteine (C), Aspartic acid (D), Glutamic acid (E) or Arginine (R);
(7) the residue Histidine (H) may be substituted by Aspartic acid (D), Leucine (L), Asparagine (N), Proline (P), Glutamine (Q), Arginine (R) or Tyrosine (Y);
(8) the residue Isoleucine (I) may be substituted by Glutamic acid (F), Leucine (L), Methionine (M), Asparagine (N) or Valine (V);
(9) the residue Lysine (K) may be substituted by Glutamic acid (E), Methionine (M), Asparagine (N), Glutamine (Q), Arginine (R) or Threonine (T);
(10) the residue Leucine (L) may be substituted by Glutamic acid (F), Histidine (H), Isoleucine (I), Methionine (M), Proline (P), Glutamine (Q), Arginine (R), Valine (V) or Tryptophan (W);
(11) the residue Methionine (M) may be substituted by Isoleucine (I), Lysine (K), Leucine (L), Arginine (R), Threonine (T) or Valine (V);
(12) the residue Asparagine (N) may be substituted by Aspartic acid (D), Histidine (H), Isoleucine (I), Lysine (K), Serine (S), Threonine (T) or Tyrosine (Y);
(13) the residue Proline (P) may be substituted by Histidine (H), Leucine (L), Glutamine (Q), Arginine (R) or Serine (S);
(14) the residue Glutamine (Q) may be substituted by Glutamic acid (E), Histidine (H), Lysine (K), Leucine (L), Proline (P) or Arginine (R);
(15) the residue Arginine (R) may be substituted by Cysteine (C), Glycine (G), Histidine (H), Lysine (K), Leucine (L), Methionine (M), Proline (P), Glutamine (Q), Threonine (T) or Tryptophan (W);
(16) the residue Serine (S) may be substituted by Alanine (A), Cysteine (C), Asparagine (N), Proline (P), Threonine (T), Tryptophan (W) or Tyrosine (Y);
(17) the residue Threonine (T) may be substituted by Alanine (A), Lysine (K), Methionine (M), Asparagine (N), Arginine (R) or Serine (S);
(18) the residue Valine (V) may be substituted by Aspartic acid (D), Glutamic acid (E), Isoleucine (I), Leucine (L) or Methionine (M);
(19) the residue Tryptophan (W) may be substituted by Cysteine (C), Leucine (L), Arginine (R) or Serine (S); and
(20) the residue Tyrosine (Y) may be substituted by Cysteine (C), Aspartic acid (D), Glutamic acid (F), Histidine (H), Asparagine (N), or Serine (S).

18. The polypeptide DeLCify of claim 1, which is DeLCify-1 having the amino acid sequence set forth in SEQ ID NO: 6, DeLCify-2 having the amino acid sequence set forth in SEQ ID NO: 7 or DeLCify-3 having the amino acid sequence set forth in SEQ ID NO: 8.

19. A pharmaceutical composition, comprising the polypeptide DeLCify set forth in claim 1, and a pharmaceutically acceptable carrier.

20. A method for treating a LPC-related disease or order in a subject comprising administering to the subject a therapeutically effective amount of a therapeutically effective amount of the polypeptide DeLCify set forth in claim 1.


SEQUENCE LISTING (8 sequences)

SEQ ID NO: 1 - protein, 491 aa, synthetic construct
SGYHIGVGRADCTGQVADINLMGYGKSGQNAQGILTRLYSRAFIMAEPDGSNRTVFVSIDIGMVSQRLRLEVLNRLQSKYGSLYRRDNVILSGTHTHSGPAGYFQYTVFVIASEGFSNQTFQHMVTGILKSIDIAHTNMKPGKIFINKGNVDGVQINRSPYSYLQNPQSERARYSSNTDKEMIVLKMVDLNGDDLGLISWFAIHPVSMNNSNHLVNSDNVGYASYLLEQEKNKGYLPGQGPFVAAFASSNLGDVSPNILGPRCINTGESCDNANSTCPIGGPSMCIAKGPGQDMFDSTQIIGRAMYQRAKSKTCKPALGYSFAAGTIDGVGGLNFTQGKTEGDPFWDTIRDQILGKPSEEIKECHKPKPILLHTGELSKPHPWHPDIVDVQIITLGSLAITAIPGEFTTMSGRRLREAVQAEFASHGMQNMTVVISGLCNVYTHYITTYEEYQAQRYEAASTIYGPHTLSAYIQLFRNLAKAIATDTVANL

SEQ ID NO: 2 - protein, 99 aa, synthetic construct
MAKRTFSNLETFLIFLLVMMSAITVALLSLLFITSGTIENHKDLGGHFFSTTQSPPATQGSTAAQRSTATQHSTATQSSTATQTSPVPLTPESPLFQNF

SEQ ID NO: 3 - protein, 12 aa, synthetic construct
MPLFQMPLFQNF

SEQ ID NO: 4 - protein, 155 aa, synthetic construct
SRGPEPPFFKQLIVPLIPSIVDRAPKGRTFGDVLQPAKPEYRVGEVAEVIFVGANPKNSVQNQTHQTFLTVEKYEATSTSWQIVCNDASWETRFYWHKGLLGLSNATVEWHIPDTAQPGIYRIRYFGHNRKQDILKPAVILSFEGTSPAFEVVTI

SEQ ID NO: 5 - protein, 145 aa, synthetic construct
SRGPEPPFFKQLIVPLIPSIVDRAPKGRTFGDVLQPAKPEYRVGEVAEVIFVGANPKNSVQNQTHQTFLTVEKYEATSTSWQIVCNDASWETRFYWHKGLLGLSNATVEWHIPDTAQPGIYRIRYFGHNRKQDILKPAVILSFEG

SEQ ID NO: 6 - protein, 745 aa, synthetic construct
MAKRTFSNLETFLIFLLVMMSAITVALLSLLFITSGTIENHKDLGGHFFSTTQSPPATQGSTAAQRSTATQHSTATQSSTATQTSPVPLTPESPLFQNFSGYHIGVGRADCTGQVADINLMGYGKSGQNAQGILTRLYSRAFIMAEPDGSNRTVFVSIDIGMVSQRLRLEVLNRLQSKYGSLYRRDNVILSGTHTHSGPAGYFQYTVFVIASEGFSNQTFQHMVTGILKSIDIAHTNMKPGKIFINKGNVDGVQINRSPYSYLQNPQSERARYSSNTDKEMIVLKMVDLNGDDLGLISWFAIHPVSMNNSNHLVNSDNVGYASYLLEQEKNKGYLPGQGPFVAAFASSNLGDVSPNILGPRCINTGESCDNANSTCPIGGPSMCIAKGPGQDMFDSTQIIGRAMYQRAKSKTCKPALGYSFAAGTIDGVGGLNFTQGKTEGDPFWDTIRDQILGKPSEEIKECHKPKPILLHTGELSKPHPWHPDIVDVQIITLGSLAITAIPGEFTTMSGRRLREAVQAEFASHGMQNMTVVISGLCNVYTHYITTYEEYQAQRYEAASTIYGPHTLSAYIQLFRNLAKAIATDTVANLSRGPEPPFFKQLIVPLIPSIVDRAPKGRTFGDVLQPAKPEYRVGEVAEVIFVGANPKNSVQNQTHQTFLTVEKYEATSTSWQIVCNDASWETRFYWHKGLLGLSNATVEWHIPDTAQPGIYRIRYFGHNRKQDILKPAVILSFEGTSPAFEVVTI

SEQ ID NO: 7 - protein, 653 aa, synthetic construct
MPLFQNFSGYHIGVGRADCTGQVADINLMGYGKSGQNAQGILTRLYSRAFIMAEPDGSNRTVFVSIDIGMVSQRLRLEVLNRLQSKYGSLYRRDNVILSGTHTHSGPAGYFQYTVFVIASEGFSNQTFQHMVTGILKSIDIAHTNMKPGKIFINKGNVDGVQINRSPYSYLQNPQSERARYSSNTDKEMIVLKMVDLNGDDLGLISWFAIHPVSMNNSNHLVNSDNVGYASYLLEQEKNKGYLPGQGPFVAAFASSNLGDVSPNILGPRCINTGESCDNANSTCPIGGPSMCIAKGPGQDMFDSTQIIGRAMYQRAKSKTCKPALGYSFAAGTIDGVGGLNFTQGKTEGDPFWDTIRDQILGKPSEEIKECHKPKPILLHTGELSKPHPWHPDIVDVQIITLGSLAITAIPGEFTTMSGRRLREAVQAEFASHGMQNMTVVISGLCNVYTHYITTYEEYQAQRYEAASTIYGPHTLSAYIQLFRNLAKAIATDTVANLSRGPEPPFFKQLIVPLIPSIVDRAPKGRTFGDVLQPAKPEYRVGEVAEVIFVGANPKNSVQNQTHQTFLTVEKYEATSTSWQIVCNDASWETRFYWHKGLLGLSNATVEWHIPDTAQPGIYRIRYFGHNRKQDILKPAVILSFEGTSPAFEVVTI

SEQ ID NO: 8 - protein, 643 aa, synthetic construct
MPLFQNFSGYHIGVGRADCTGQVADINLMGYGKSGQNAQGILTRLYSRAFIMAEPDGSNRTVFVSIDIGMVSQRLRLEVLNRLQSKYGSLYRRDNVILSGTHTHSGPAGYFQYTVFVIASEGFSNQTFQHMVTGILKSIDIAHTNMKPGKIFINKGNVDGVQINRSPYSYLQNPQSERARYSSNTDKEMIVLKMVDLNGDDLGLISWFAIHPVSMNNSNHLVNSDNVGYASYLLEQEKNKGYLPGQGPFVAAFASSNLGDVSPNILGPRCINTGESCDNANSTCPIGGPSMCIAKGPGQDMFDSTQIIGRAMYQRAKSKTCKPALGYSFAAGTIDGVGGLNFTQGKTEGDPFWDTIRDQILGKPSEEIKECHKPKPILLHTGELSKPHPWHPDIVDVQIITLGSLAITAIPGEFTTMSGRRLREAVQAEFASHGMQNMTVVISGLCNVYTHYITTYEEYQAQRYEAASTIYGPHTLSAYIQLFRNLAKAIATDTVANLSRGPEPPFFKQLIVPLIPSIVDRAPKGRTFGDVLQPAKPEYRVGEVAEVIFVGANPKNSVQNQTHQTFLTVEKYEATSTSWQIVCNDASWETRFYWHKGLLGLSNATVEWHIPDTAQPGIYRIRYFGHNRKQDILKPAVILSFEG